(19) United States
(12) Patent Application Publication
(10) Pub. No.: US 2015/0328169 A1
(43) Pub. Date: Nov. 19, 2015

(54) POLYMERIC PARTICLES-BASED TEMOZOLOMIDE DOSAGE FORM

(71) Applicants: OY FILANA LTD, Aura (FI); UNICHEMPHARM LTD, Edinburgh (GB)

(72) Inventors: Ekaterina VASILENKO, Sarov (RU); Evgeny VORONTSOV, Moscow (RU); Evgenij SEVERIN, Moscow (RU); Victor GULENKO, Moscow region (RU); Maxim MITROKHIN, Moscow (RU); Maksim IURCHENKO, Kiev (UA)

(73) Assignees: UNICHEMPHARM LTD, Edinburgh (US); OY FILANA LTD, Aura (FI)

(21) Appl. No.: 14/650,799

(22) PCT Filed: Dec. 10, 2013
(86) PCT No.: PCT/FI2013/051151

§ 371 Date: Jun. 9, 2015

Related U.S. Application Data
(60) Provisional application No. 61/735,089, filed on Dec. 10, 2012.

(30) Foreign Application Priority Data
Dec. 10, 2012 (FI) 20126281

(51) Int. Cl. A61K 9/51; A61K 31/495

(52) U.S. Cl. CPC A61K 9/5153; A61K 31/495

(57) ABSTRACT

The invention relates to pharmacology and medicine, and more specifically to slow-release antitumor drug composition based on biodegradable poly(lactic-co-glycolic acid) (PLGA). The composition comprises temozolomide (TMZ) as the active ingredient and comprises, in addition, surface-active material and a cryoprotectant as parts of nanoparticles.

### FIELD OF THE INVENTION

The present invention relates to the field of pharmacology and medicine, specifically of antitumor drugs based on poly(lactic-co-glycolic acid)(PLGA).

### BACKGROUND OF THE INVENTION

Melanoma is a high-grade tumor, which makes 1-4% of all oncologic diseases, and is marked up by high rate of metastases.

Primary tumors in brain and brain metastases are found in 14 incidences per 100 thousand people, the mortality rate whereof reaching 10.8 incidences per 100 thousand people (the second place at central nervous system pathologies after head-and-brain injuries), see also: Ulitin A. Yu., Olyushin V. Ye., Polyakov I. V. Epidemiologiya pervichykh opkholey golovnogo mozga v Sankt-Peterburge. [In Russian: Epidemiology of Primary Brain Tumors in St. Petersburg] No. 1. 2005. p. 610; American Cancer Society: Cancer Facts and Figures 2006/Atlanta, Ga.: American Cancer Society, 2006/Also available online. Last accessed Feb. 14, 2006; Behin A. Primary brain tumors in adults/Lancet Seminar. 2003. V. 361. pp. 323-331; CBTRUS: Statistical Report: Primary Brain Tumors in the United States, 1995-1999. (http://www.cbtrus.org./reports)/2002/2002 report.pdf) {Accessed 19, October 2005}; Legler J. M., Gloeckler Ries L. A., Smith M. A. et al. Brain and other central nervous system cancers: recent trends in incidence and mortality/J. Natl. Cancer Inst. 1999. V. 91. pp. 2050A-51.

One of the leading components in the modern standards of treating the metastasizing melanoma and brain tumors is temozolomide (international generic name) in such drugs as Temodal®, Temodar®; see also: Stevens M. F. G. et al. Antitumor activity and pharmacokinetics in mice of 8-carbamoyl-3-methyl-imidazo[5,1-d]-1,2,3,5-tetrazin-4-(3H)-one (CCPG 81045; M&B 39831), a novel drug with potential as an alternative to dacarbazine/Cancer. Res. 1987. V. 47. pp. 5846-5852.

Temozolomide (TMZ) belongs to the group of the second-generation ankylating (antineoplastic chemotherapeutic) agents named imidazole tetrazines. Temozolomide is characterized by a wide range of antitumor activities: It is active against malignant melanoma, mycosis fungoides, and advanced glioma. Tests in vitro provide evidence that TMZ is also active against ovarian tumors and a number of other tumors resistant to drugs applied, such as dacarbazine, carmustine, cisplatin, doxorubicin, 5-fluorouracil, etoposide, and vinblastine.

Currently, the medical compositions containing TMZ are manufactured as capsules for oral use. They are prescribed for adults and for children above three years. The maximal length of treatment is two years. Temozolomide does not provide any irritant action upon the gastrointestinal tract mucosa, and thus it is suitable for oral use. This dosage form is very patient-friendly. However, it is difficult for the medical staff to control the course of therapy.

Clinical trials have shown that TMZ was absorbed very fast and reached Cmax in 0.7 hours, and had the half lifetime of only 1.8 h. Temozolomide demonstrates a good distribution in all tissues, including penetrating through the brain-blood barrier [Radulesku G. G. Temodal—novyi protivoopukholevyi preparat dlya lecheniya zlokachestvennykh gliom [In Russian: Temodal—a New Antitumor Drug for the Treatment of Malignant Gliomas]/Terra Medica nova. 2002. #3]. However, the TMZ concentration in plasma decreases fast upon the drug administration. That is why multiple administrations are necessary for keeping the efficient drug concentration in blood, which causes essential inconveniences for patients.

Temozolomide, like most antitumor drugs, has a number of side effects affecting digestion system (nausea, vomiting, constipation, anorexia, diarrhea, abdominal pains, dyspepsia, taste disorders), central nervous system (fatigue, headache, drowsiness, dizziness, paresthesia), skin (cutaneous eruption, alopecia, skin itching), respiratory system (dyspnea), blood (thrombocytopenia and neutropenia Grade 3 or 4, pancytopenia, leukopenia and anemia) [Temozolomide Description/Internet version of “Klifar” (www.drugreg.ru)]. Other side effects are fever, asthenia, body weight loss, cacesthesia and chill. Using TMZ is contraindicative at bad myelosuppression, pregnancy, lactation, hypersensitivity to temozolomide or to dacarbazine. Temozolomide may cause sleepiness and fatigue feeling, so it negatively affects the ability to drive.

Various publications exist which disclose pharmaceutical compositions in which a therapeutically active agent is included in nanoparticles. For instance, Cheng, J. et al. (Formulation of functionalized PLGA-PEG nanoparticles for in vivo targeted drug delivery. Biomaterials, 2007 Vol. 28 (5). p. 869-876) studied docetaxel and A10 Aptamer administration in PLGA-b-PEG-COOH nanoparticles altering the formulation parameters, and achieved a tumor-specific systemic targeting of a NP-Apt bioconjugate system in vivo. Vranckx et al. (U.S. Pat. No. 5,500,224, Pharmaceutical compositions containing nanocapsules) describe poly-2-alkyl-cyanoacrylate (PACA) nanocapsules containing aqueous solution or suspension of the therapeutically active agent (e.g. calcitonin, somatostatin, insulin or heparin). The size of the nanocapsules is under 500 nm.

Since temozolomide is a relatively new medicament, there are only few publications which disclose compositions thereof with polymers. One of such publications is the work described by the scientists from the University of Tennessee, USA [Akbar U., Jones T., Winestone J. et al. Delivery of temozolomide to the tumor bed via biodegradable gel matrices in a novel model of intracranial glioma with resection/J. Neurooncol. 2009. V. 94 (2). pp. 203-212]. The authors added temozolomide to a PLGA-based gel, using polyethyleneglycol 400, N-methylpyrrolidone, triethyl citrate and acetyl triethyl citrate as plasticizers. The composition was administered locally, i.e. it was injected intracranially into the post-resection cavity after resection of the tumor. In the experiments on animals, prolonged action of the above composition was shown (over 30 days).

A publication of Chinese scientists describes obtaining microparticles based on PLGA 75:25 with TMZ added [Zhang H., Gao S. Temozolomide/PLGA microparticles and antitumor activity against Glioma C6 cancer cells in vitro/Int. J. Pharm. 2007. V. 329. pp. 122-128]. During their experiments, the authors obtained large particles sized from 50 to 80 μm, the drug sorption level being rather high (about 80%). The polymer was also shown to be an inert matter that did not affect the morphology or proliferating activities of cells. The medicament's prolonged (during 3 days) dose-dependent cytotoxic effect upon glioma C6 cancer cells was also proved (cell proliferation inhibition).

It should be noted that, as compared to the present invention, the above-referenced polymeric temozolomide composition by Zhang and Gao represents micro-sized particles. The method of obtaining said composition, as well as the composition itself, are notable for their low-level manufacturability resulting from the unreasonably high and no-purpose consumption of the medicament (TMZ) and surface-active material; no widespread investigation was conducted regarding the antitumor activity of the composition obtained, except for glioma C-6 in vitro; no data on the toxic action of the microsized composition, particularly upon the blood components, are available.

### SUMMARY OF THE INVENTION

The invention is focused on solving problems relating to the toxicity of the active agent—temozolomide, to the contraindications thereof, as well as to prolonging its action.

The efficacy of said medicament needs to be increased and, therefore, its curative dose and its toxic action must be reduced.

The above tasks can be solved by developing nano-sized medicament forms based on biodegradable polymers, and comprising temozolomide as the active ingredient.

The development process resulted in a pharmaceutical composition, which comprises temozolomide as an active ingredient, as well as a biodegradable polymer, a surface-active material and a cryoprotectant, the component ratios (% wt) being as follows:

| | |
|---|---|
| temozolomide | 10-20 |
| biodegradable polymer | 65-80 |
| surface-active material | 2-3 |
| cryoprotectant | up to 100% wt, |

as parts of nanoparticles.

The biodegradable polymer represents a poly(lactic-co-glycolic acid) (PLGA), molar ratio 50:50, or a PLGA copolymer, molar ratio 75:25, or a PLGA copolymer with a free carboxyl group (PLGA-COOH), molar ratio 50:50.

The surface-active material represents polyvinyl alcohol or serum albumin.

The cryoprotectant represents D-mannitol or glucose.

The size of the nanoparticles is 200-500 nm.

The pharmaceutical composition according to the present invention is an antitumor drug composition, which is specifically useful in the treatment of malignant neoplasms.

The nanoparticles comprising temozolomide as the active ingredient may be manufactured as dosage forms for oral use, such as tablets or capsules, and can be used, under controlling the peripheral blood leukocyte level, in courses until the malignant neoplasms are eliminated.

The nanoparticles comprising temozolomide as the active ingredient can also be included into a sterile suspension containing water-salt solution for intravenous injections, which may be administered under controlling the peripheral blood leukocyte level, in courses until the malignant neoplasms are eliminated.

### BRIEF DESCRIPTION OF THE DRAWINGS

The essence of the invention and the possibility to obtain the technical result will be clearer from the following description containing the references to drawings accompanying it.

FIG. 1 shows a diagram representing the increase in the efficacy of temozolomide when used as a part of a composition based on PLGA 50:50 (TMZ-PLGA 50/50) regarding B16 mouse melanoma cells in vitro.

FIG. 2 is a diagram demonstrating the increase in efficacy of temozolomide when used as a part of a composition based on PLGA 50:50 (TMZ-PLGA 50/50) regarding C6 rat glioma cells in vitro.

FIG. 3 shows the increase in the efficacy of temozolomide when used as a part of a composition based on PLGA 50:50 (TMZ-PLGA 50/50) regarding Mel-10 human melanoma cells in vitro.

FIG. 4 shows the increase in the efficacy of temozolomide when used as a part of a composition based on PLGA 50:50 (TMZ-PLGA 50/50) regarding U377MG human glioma cells in vitro.

FIG. 5 demonstrates the tumor growth dynamics in control mice upon the inoculation of B16 melanoma tumor cells (control) and in experimental mice treated with free temozolomide dosed as 60 mg/kg and with temozolomide as a part of a composition based on PLGA 50:50 (TMZ-PLGA 50/50) dosed as 60 mg/kg, at daily drug administration within 9 days starting from the day following the day of the tumor inoculation.

FIG. 6 shows the dimensions upon the B16 melanoma tumor inoculation at treating the mice with free temozolomide dosed as 40 mg/kg and with temozolomide as a part of a composition based on PLGA 50:50 (TMZ-PLGA 50/50) dosed as 40 mg/kg on the 5^{th} day upon the tumor inoculation dosed as 1 million of tumor cells per mouse when administering the medicaments starting from the second day upon the day of the tumor inoculation within 9 days.

FIGS. 7A and 7B show the dimensions of B16 melanoma tumor when treating the mice with free temozolomide dosed as 60 mg/kg and with temozolomide as a part of a composition based on PLGA 50:50 (TMZ-PLGA 50/50) dosed as 60 mg/kg on the 10^{th} day (A) and on the 16^{th} day (B) upon the tumor inoculation dosed as 1 million of tumor cells per mouse when administering the medicaments starting from the second day upon the day of the tumor inoculation within 9 days.

FIGS. 8A and 8B show the inhibition of B16 melanoma tumor growth when treating mice with free temozolomide and with temozolomide as a part of a composition based on PLGA 50:50 (TMZ-PLGA 50/50) dosed as 40 mg/kg (A) and 60 mg/kg (B) in dynamics, upon the tumor inoculation dosed as 1 million of tumor cells per mouse. The arrows indicate the day of drug administration (drugs were administered daily for 9 days, starting 2 days after tumor inoculation).

FIG. 9 shows the dynamics of deaths of mice inoculated with B16 melanoma tumor upon the treatment with temozolomide as a part of a composition based on PLGA 50:50 (TMZ-PLGA 50/50) dosed as 40 mg/kg, as compared to mice treated with free temozolomide dosed as 40 mg/kg upon the tumor inoculation dosed as 1 million of tumor cells per mouse. Drugs were administered daily for 9 days, starting 2 days after tumor inoculation. Increase in lifespan was 18.1%.

FIG. 10 shows the dynamics of peripheral blood leukocytes count changes in control mice upon the inoculation of B16 melanoma tumor (control) and in experimental mice treated with free temozolomide dosed as 40 mg/kg and with temozolomide as a part of a composition based on PLGA 50:50 (TMZ-PLGA 50/50) dosed as 40 and 60 mg/kg.

### DETAILED DESCRIPTION OF THE INVENTION

The advisability of using nanosomal systems for the treatment of malignant neoplasms is determined by the possibility to perform the targeted transport of medicaments into the tumor, which is, in its turn determined by the features of tumor tissues: The enhanced permeability of capillaries feeding the tumor, and lymphatic drainage disruption. These features create the EPR effect—the effect of Enhanced Permeability and Retention of particles within the tumor, which promotes the penetration and accumulation of particles within the tumor.

A great advantage of this technique is its flexibility. Depending on the nature and on the location of the tumor, the carriers' properties can be changed, and predominant drug localization in one or another organ/tissue can be obtained. Besides, modifying the particle surface (the type and the concentration of the surface-active material used) also allows to purposefully change pharmacokinetics and nanoparticle distribution in the body depending on the location of the tumor to be treated.

For developing an antitumor drug, biodegradable poly(lactic-co-glycolic acids) are advantageous.

These copolymers are biocompatible, non-toxic and non-immunogenic, and they are the few ones permitted for being used in developing medicaments for intravenous administration. A great advantage of PLGAs is their ability to increase the efficacy of drugs. Medicaments based on these polymers are also characterized by all the above listed positive effects: passive targeted transport and reduced toxicity provided thereby, longer action, and the ability to overcome drug resistance.

The nano-sized drug compositions comprising temozolomide can be used both orally as capsules or tablets and as injections.

The technical result of the present invention can be reached by adding the medicament to nanoparticles obtained on the basis of commercially available biodegradable polymers. As such, the poly(lactic-co-glycolic acids) (PLGA 50:50 and PLGA 75:25) and a poly(lactic-co-glycolic acid) with the free carboxyl group (PLGA-COOH 50:50) may be used.

To obtain the nanoparticles, the polymers having the molecular mass ranging from of 10 to 300 kDa and the molar ratio of lactic/glycolic acid residues ranging from 25:75% to 50:50% are used. To obtain a stable pharmaceutical formula representing nanoparticles sized 200-500 nm and having prolonged release of the drug, surface-active materials, such as polyvinyl alcohol (PVA) or serum albumin, and cryoprotectants, such as D-mannitol or glucose, are also used. The drug composition is obtained by the single-stage emulsification technique (water/oil). The drug sorption within the nanoparticles takes place when removing organic solvent from the emulsion obtained.

A new technical result is obtained with the newly developed drug composition. In the present specification the form and composition of the polymeric particles containing temozolomide are shown. The high specific activity thereof is proved in in vitro and in vivo experiments; a wide range of antitumor activity is shown in in vitro experiments; and a prolonged action at the reduced toxic effect on blood components is also shown.

Consequently, it is concluded that a new technical result has been achieved with the drug composition according to the invention. It has been shown that said drug composition in the form of polymeric particles based on the anticancer agent temozolomide has durable action and higher therapeutic efficacy at lower toxicity, as compared to the drug substance itself. These results also allow making the conclusion that using said drug composition the safety margins are higher and the curative ratio may be increased despite of decreasing dosage frequency.

The invention is further illustrated by the examples below.

### Example 1

### Obtaining Polymeric Particles Containing Temozolomide

The temozolomide substance taken as 20% wt from the PLGA used was added to 9 ml of polymer solution (800-1200 mg) in dichloromethane while stirring with magnetic stirrer for 5 minutes. The suspension was stirred for another 20-30 minutes, and added at intensive 2-minute stirring to the 2%-PVA-water solution saturated with temozolomide (40-50 ml) or to 1%-albumin-water solution (40-50 ml). The mixture obtained was intensively stirred for another 30 minutes and then homogenized with Ultra-Turrax T-25 (IKA®, Germany) with S25N-25F attachment at 24 thousand rpm three times, 1 minute each, with two breaks, 1 minute each. A foamed emulsion of the “oil-in-water” type (0/W) was obtained. The emulsion was stirred for 2 h at room temperature under exhaust-duct ventilation until the organic solvent was completely removed. The suspension obtained was settled by centrifuging on Beckman J2-21 (USA) at 12 thousand rpm within 30 minutes. The supernatant was carefully sucked out using a pipettor, 10 ml of water were added to the remaining residues and carefully mixed using a spatula, and then the residues were ground. After that, the mixture was re-homogenized using the same machine with the S25N-10G attachment in the same mode as previously. D-mannitol or glucose (18-22 mg), as a cryoprotectant, was added to the colloidal solution obtained; then the content was transferred to a round-bottomed flask, frozen, and lyophilized during 20-24 h at 0.1-0.8 mbar. The product obtained was sterilized by γ-radiation dosed as 22 kGy.

Average particle size found by photon correlation spectroscopy on Coulter N4MD (USA), the dynamic light scattering analyzer, was 200-500 nm, which contributes to efficient absorption in gastrointestinal tract. The size of the particles obtained depends on the polymer type, emulsion stabilizer, their concentration, as well as on the homogenization conditions.

| Composition 1 (on Example 1) | % wt |
|---|---|
| Polymer (PLGA 50:50) | 72.0 |
| Temozolomide | 17.0 |
| PVA (polyvinyl alcohol) | 2.7 |
| Cryoprotectant (D-Mannitol) | 8.3 |
| Size of particles | 378 ± 84 nm |

| Composition 2 (on Example 1) | % wt |
|---|---|
| Polymer (PLGA 75:25) | 71.1 |
| Temozolomide | 17.4 |
| PVA (polyvinyl alcohol) | 3.2 |
| Cryoprotectant (glucose) | 8.3 |
| Size of particles | 281 ± 73 nm |

| Composition 3 (on Example 1) | % wt |
|---|---|
| Polymer (PLGA-COOH 50:50) | 73.1 |
| Temozolomide | 16.7 |
| Surface-active material (albumin) | 3.5 |
| Cryoprotectant (D-Mannitol) | 10.9 |
| Size of particles | 324 ± 57 nm |

The temozolomide-PLGA drug composition obtained as described above, in form of a sterile salt-water suspension, may be administered daily to the patient intravenously, under the control of peripheral blood leukocytes, in courses, until the malignant neoplasms are eliminated.

### Example 2

### Estimation of the Antitumor Activity of the Temozolomide Substance and of Temozolomide as the Particles of a Polymeric Composition Based on PLGA 50:50, Obtained as in Example 1 (Composition 1), In Vitro Regarding Various Human and Animal Tumor Cell Lines

Temozolomide is known to be highly efficient regarding such malignant neoplasms as melanoma and glioma. This is why the following human tumor lines were taken as experimental models: B16 mouse melanoma and C6 rat glioma; and the following human tumor cell lines: Mel-10 melanoma and U377MG glioma.

The cells of the lines to be inoculated were cultivated in DMEM (Sigma) containing 10% of fetal bovine serum (Gibco) and 50 μg/ml of gentamicin (ICN) in plastic cultural flasks (Corning-Costar). The antitumor activity of free temozolomide and that of the nanoform of temozolomide were estimated through MTT-test using Mosmann technique [Mossman T. Rapid colorimetric assay for cellular growth and survival: Application to proliferation and cytotoxicity assays/J. Immunol. Meth. 1983. V. 65 (1-2). pp. 55-63]. The cells were inoculated into 96-well plates, 5-7 thousand cells per well one day before adding the medicaments. Various doses of the medicaments under test were added to the cells one time and incubated in standard cultivating conditions for 72 hours. The vitality of the cells upon incubating together with antitumor medicaments was estimated using the MTT test. For this purpose, 4 h prior to the end of the incubation, 50 μl of MTT assay (Sigma) in the concentration of 1 mg/ml in the cell cultivating medium were added to each well. Upon coloring, the medium was removed, the formazan crystals laid-out were dissolved in 100 μl of DMSO, and the color intensity was measured by sorption at 540 nm using desktop spectrophotometer Labsystem (Finland). Cell survival was evaluated in percents of the untreated control; and cell survival curves were used to calculate the value of IC₅₀—the drug concentration at which the death of 50% of cells is observed.

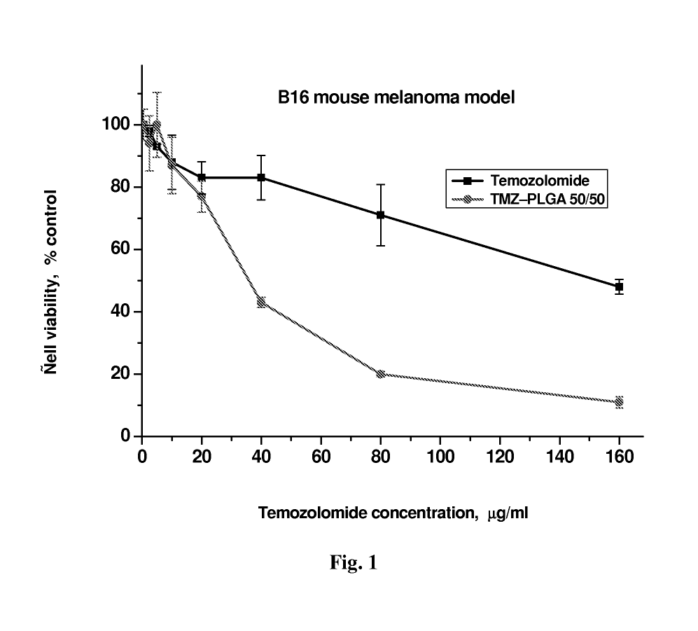

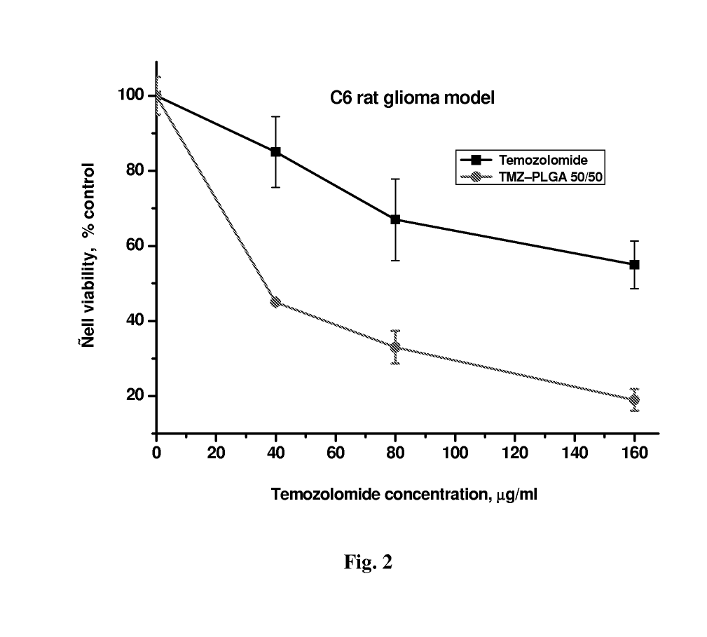

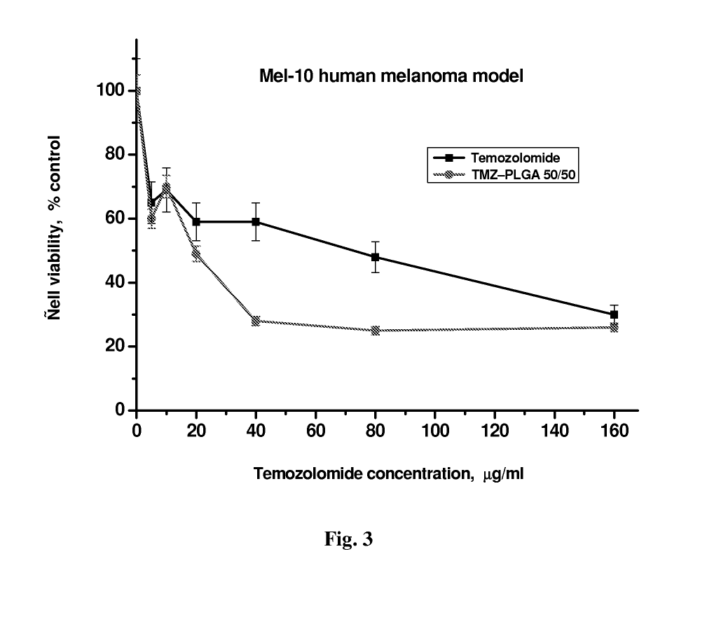

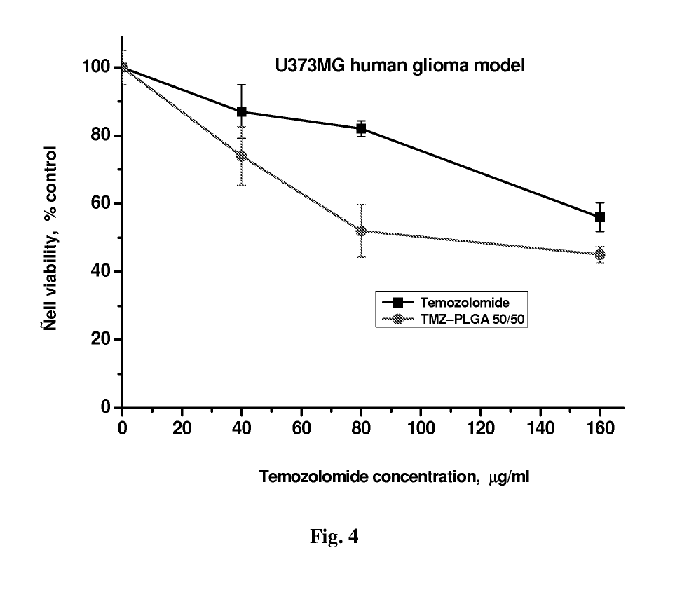

The results obtained are shown in FIGS. 1-4. It follows from the results presented that in all the models above, temozolomide in form of the nanoparticles of a composition containing PLGA 50:50 was more efficient regarding tumor cells than the free medicament.

### Example 3

### Estimation of the Antitumor Activity of the Temozolomide Substance and of Temozolomide as the Particles of a Polymeric Composition Based on PLGA 50:50, Obtained as in Example 1 (Composition 1), In Vivo Relating to a Solid Tumor in a Mouse

The experiment was performed on female C₅₇Balb/c mice weighing 22-24 g. The mice were kept in standard cages in groups of 10 animals in each, in the conditions of unlimited access to water and food, at natural lighting changes, at the temperature of 20-22° C. and humidity of 75%. Testing the medicament started upon a two-week isolation period of the animals. The groups of test animals consisted of 10 mice each, the experiments being reproduced 2-3 times. The experimental model was a solid tumor of B-16 mouse melanoma. The tumors were inoculated in accordance with standard methods [Treshchalina E. M., Zhukova O. S., Gerasimova G. K., et al. Metodicheskiye ukazaniya po isucheniyu protivoopukholevoy aktivnosti farmakologicheskikh veshchestv [In Russian: Methodical Recommendations on Studying the Antitumor Activity of Pharmaceuticals]/Iz Knigi “Rukovodstvo po eksperimentalnomu (doklinicheskomu) isucheniyu novykh farmakologicheskikh veshchestv” [In Russian: From the Book titled: Instructions on Experimental (Preclinical) Studying New Pharmaceuticals]/Pod red. R. U. Khabriyeva, 2 izd, M.: Meditsina, 2005, p. 637-651], subcutaneously, in the subscapular area, in the amount of 10⁶ cells. The treatment started in 24 hours thereupon. Injections were made intraperitoneally, once a day for 9 days. Dosages: group (gr.) No. 1 (control)—0.2 ml of physiological (phys.) solution; gr. No. 2 (comparative drug)—40 mg/kg of temozolomide in 0.2 ml of phys. solution; gr. No. 3 of temozolomide in nanoparticles of a composition based on PLGA 50:50-40 mg/kg in 0.2 ml of phys. solution; gr. No. 4 (comparative drug)—60 mg/kg of temozolomide in 0.2 ml of phys. solution; gr. No. 5 temozolomide as a part of a composition based on PLGA 50:50-60 mg/kg in 0.2 ml of phys. solution.

The antitumor activities of the medicaments tested were estimated on the basis of comparing the tumor growth kinetics in the groups of treated and control animals. To study the kinetics of tumor growth, the two mutually perpendicular dimensions of a tumor node were measured during the entire period of tumor growth. The tumor volume was calculated in accordance with the formula accepted for ellipsoids V=ab²/2, where a—length, b—width and height of the tumor node. Tumor mass correlates with its volume, since the tumor tissue density is considered to be equal to 1 g/cm³ [Treshchalina E. M. et al., ibid].

To estimate the antitumor effect of the medicaments, the standard indicator of tumor growth inhibition (TGI, %) was used, that is descriptive of changes in the average tumor weight (P) as affected by the test preparation in treated animals (T) as compared to control animals (C) and defined as TGI=(PC−PT)/PC×100%. It is also important to note that TGI>50% is traditionally considered significant [Treshchalina E. M. et al., ibid]. To estimate toxic reactions to the administration of medicaments, the peripheral blood leukocytes were counted in dynamics during and upon the administration of the medicaments. The results obtained are presented as relevant drawings.

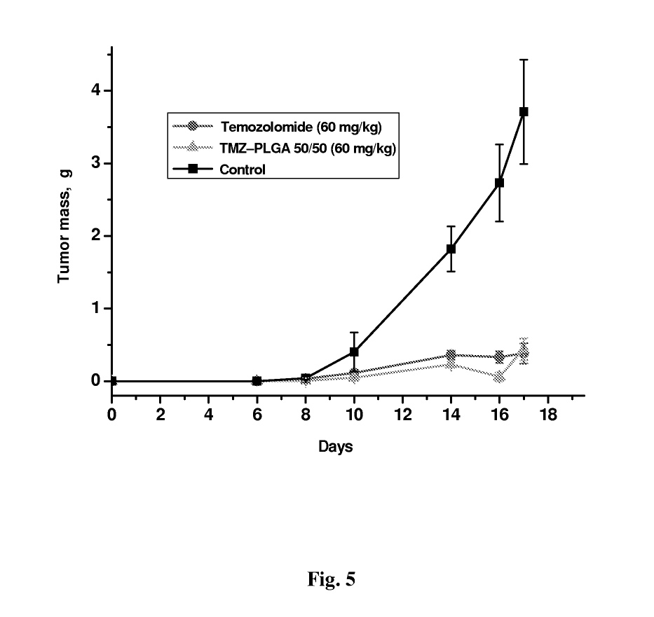

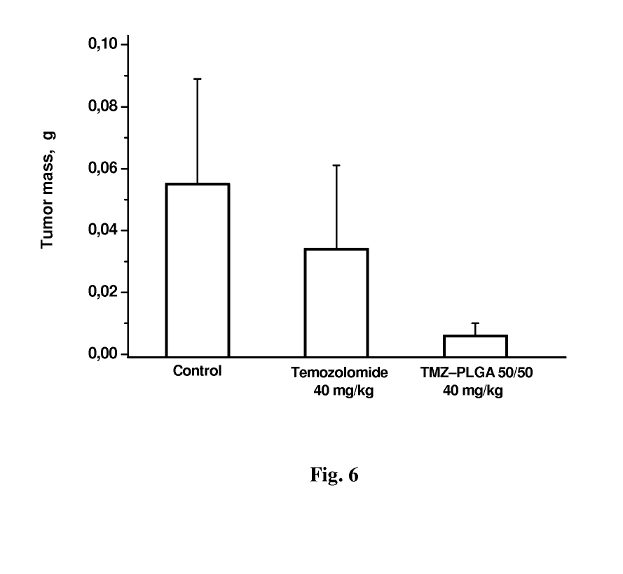

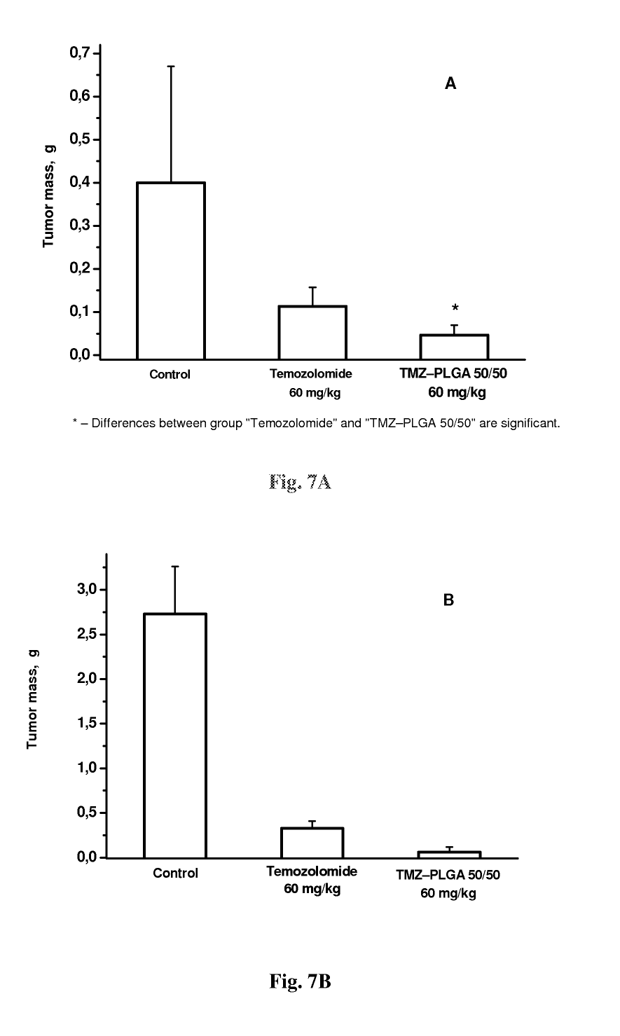

It follows from FIG. 5, FIG. 6 and FIG. 7 that temozolomide as a part of a polymeric composition based on PLGA 50:50 within the period from the 5^{th} day (when tumors appeared) through the 13^{th} day (4 days after withdrawal) inhibits the tumor growth more efficiently than the free medicament. Most efficient tumor growth inhibition was observed when using nanoparticles of the temozolomide-PLGA 50:50 composition dosed as 60 mg/kg.

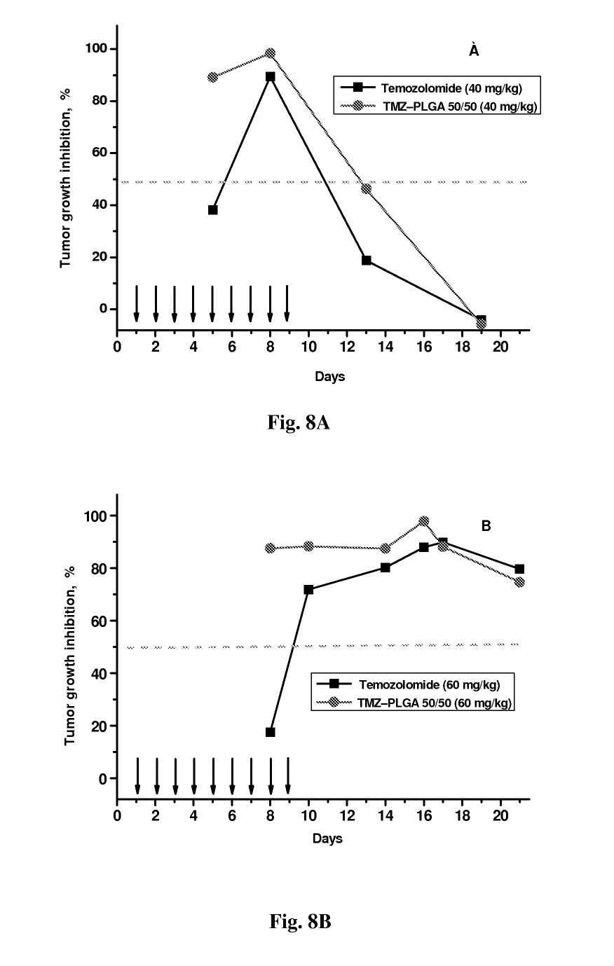

FIGS. 6 and 7 represent the tumor sizes on the 5^{th} day upon tumor inoculation and medicament administration (dose 40 mg/kg), and tumor sizes on the 10^{th} and on the 16^{th} day (dose 60 mg/kg), and FIG. 8 shows the values of TGI when the medicaments under research were effecting.

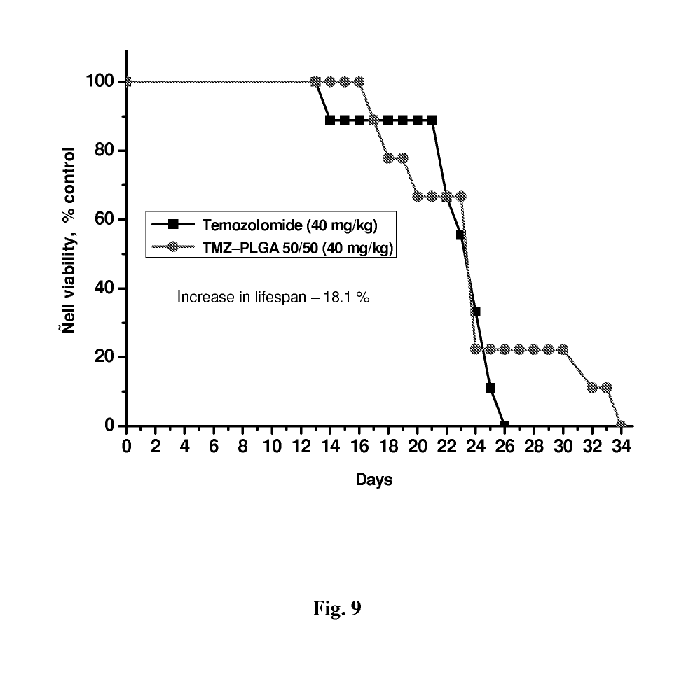

It is important to note (FIG. 8A) that free temozolomide dosed as 40 mg/kg considerably inhibits tumor growth only on the 9^{th} day of treatment (TGI is equal to 90%), and then its effect decreases fast after withdrawal. Free temozolomide dosed as 60 mg/kg (FIG. 8B) was more efficient than that dosed as 40 mg/kg, but its effect appeared later than that of using the medicament as PLGA-based nanoparticles. It is important to emphasize that temozolomide as nanoparticles within a PLGA-based composition efficiently inhibits tumor growth already on the 5^{th} day after beginning of the administration. A significant effect of medicaments as nanoparticles retains for up to 13 days when administered as 40 mg/kg of temozolomide, and for up to 20 days when administered as 60 mg/kg of temozolomide (TGI>50%). However, the increase in the average life time at using medicaments as nanoparticles based on PLGA was only observed when dosed as 40 mg/kg (FIG. 9), which is determined by the high toxicity of the medicament when used dosed as 60 mg/kg. The increase in lifespan was 18.1%.

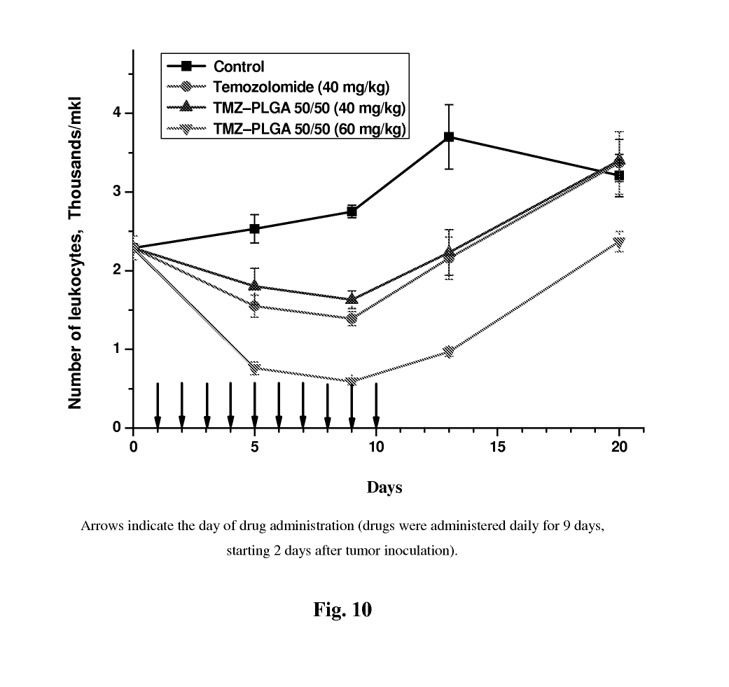

Studying the effects of the proposed drug composition on the level of the peripheral blood leukocytes in mice showed (FIG. 10) that when used in an equivalent dose (40 mg/kg), temozolomide as a part of nanoparticles containing PLGA 50:50 has even a lower toxicity than the free medicament, while the toxicity of temozolomide as a part of nanoparticles containing PLGA 50:50, dosed as 60 mg/kg, essentially increases if administered daily for 9 days. Mouse blood leukocytes were counted in the relevant groups of animals in 5, 10, 12 and 20 days upon the beginning of the experiment. Leukocytes were counted in Goryaev chamber upon diluting 10 μl of blood taken from tail vein in 40 μl of 3% acetic acid solution.

The toxicity of free temozolomide dosed as 60 mg/kg regarding the animals' peripheral blood leukocytes was similar to its toxicity at a dose of 40 mg/kg. At higher doses of temozolomide as a part of PLGA, i.e. at doses exceeding 40 mg/kg of weight, the drug administration regimen should be changed (the length of continuous administration should be reduced) in accordance with the data relating to changes in white blood cell count.

In analyzing the amount of metastases in the lungs of died mice inoculated with tumors, the reduction of metastasizing was observed when using temozolomide as a part of PLGA-based nanoparticles.

### Example 4

### Studying the Acute Toxicity of Temozolomide Substance and Temozolomide as a Part of PLGA Particles Obtained as in Example 1 (Composition 1)

The comparative analysis of the acute toxic action of the temozolomide substance and of the polymeric composition based thereon (Composition 1 as in Example 1) was performed on male and female Balb/c mice weighing 19-21 g as of the time of testing, 6 animals in each group. The mice were kept in standard cages No. 4, in the conditions of unlimited access to water and food, at natural lighting changes. Testing the toxic action of the drugs started upon a two-week isolation period of the animals. The drug was administered intraperitoneally, as a single dose. Water for injection was used as a medium. Upon the drug administration, the animals were continuously monitored for 24 hours. The total observation time was 28 days. According to the research results, the LD₅₀ values were calculated as per the state of animals on the 14^{th} day upon the administration of the drugs. The value of the mean lethal dose (LD₅₀) was determined by Litchfield-Wilcoxon method [Belenky M. L. Elementy kolichestvennoy otsenki farmakologicheskogo effekta [In Russian: Elements of Drug-Induced Effect Quantitative Estimation]/2-e izd. pererab. i dop. L.: Medgiz, 1963. p. 81-106].

The research results are presented in Tables 1 and 2 below.

TABLE 1
Acute toxicity of temozolomide and its polymeric composition
in tests on male Balb/c mice.

| Temozolomide Composition | |
|---|---|
| (Composition 1 by Example 1) | Temozolomide Substance |
| LD₁₆ 507.3 mg/kg | LD₁₆ 424.0 mg/kg |
| LD₅₀ 541.9 mg/kg | LD₅₀ 471.8 mg/kg |
| LD₈₄ 576.6 mg/kg | LD₈₄ 519.6 mg/kg |
| LD₁₀₀ 593.9 mg/kg | LD₁₀₀ 543.5 mg/kg |
| Toxic dose range was 507.3-593.9 | Toxic dose range was 424.0-543.5 |
| mg/kg | mg/kg |

TABLE 2
Acute toxicity of temozolomide and its polymeric composition
in tests on female Balb/c mice.

| Temozolomide Composition | |
|---|---|
| (Composition 1 by Example 1) | Temozolomide Substance |
| LD₁₆ 501.2 mg/kg | LD₁₆ 426.5 mg/kg |
| LD₅₀ 536.6 mg/kg | LD₅₀ 466.6 mg/kg |
| LD₈₄ 572.0 mg/kg | LD₈₄ 506.7 mg/kg |
| LD₁₀₀ 589.8 mg/kg | LD₁₀₀ 526.7 mg/kg |
| Toxic dose range was 501.2-589.8 | Toxic dose range was 426.5-526.7 |
| mg/kg | mg/kg |

Therefore, adding temozolomide to the polymeric composition based on PLGA 50/50 (Example 1, Composition 1) results in reducing its acute toxicity as compared to the primary substance.

1. A pharmaceutical composition comprising temozolomide, a biodegradable polymer, a surface-active material and a cryoprotectant, with the following component ratios, % wt:

| | |
|---|---|
| temozolomide | 10-20 |
| biodegradable polymer | 65-80 |
| surface-active material | 2-3 |
| cryoprotectant | up to 100% wt, |

as parts of nanoparticles.

2. The pharmaceutical composition according to claim 1, wherein the biodegradable polymer is a poly(lactic-co-glycolic acid) (PLGA), molar ratio 50:50, or PLGA, molar ratio 75:25, or PLGA with a free carboxyl group (PLGA-COOH), molar ratio 50:50.

3. The pharmaceutical composition according to claim 1, wherein the surface-active material is polyvinyl alcohol or serum albumin.

4. The pharmaceutical composition according to claim 1, wherein the cryoprotectant is D-mannitol or glucose.

5. The pharmaceutical composition according to claim 1, wherein the size of the nanoparticles is between 200-500 nm.

6. The pharmaceutical composition according to claim 1, wherein the nanoparticles comprising temozolomide are manufactured in oral dosage form.

7. The pharmaceutical composition according to claim 6, wherein the oral dosage form is the form of tablets or capsules.

8. The pharmaceutical composition according to claim 1, wherein the nanoparticles comprising temozolomide are included in a sterile suspension containing a water-salt solution for intravenous injection.

9. The pharmaceutical composition according to any one of claims 1 to 8 for use in treating malignant neoplasms.
